(19) United States
(12) Patent Application Publication
(10) Pub. No.: US 2016/0256462 A1
(43) Pub. Date: Sep. 8, 2016

(54) ORAL LIQUID PHARMACEUTICAL COMPOSITION OF A DPP-IV INHIBITOR

(71) Applicant: SUN PHARMACEUTICAL INDUSTRIES LIMITED, Mumbai, Maharashtra (IN)

(72) Inventors: Vinod Kumar ARORA, Gurgaon (IN); Sumit MADAN, New Delhi (IN); Rathinasabapathy VENKATESHWARAN, Madurai (IN)

(21) Appl. No.: 15/024,994

(22) PCT Filed: Sep. 24, 2014
(86) PCT No.: PCT/IB2014/064805

§ 371 Date: Mar. 25, 2016

(30) Foreign Application Priority Data
Sep. 25, 2013 (IN) 2840/DEL/2013

(51) Int. Cl. A61K 31/522; A61K 9/08; A61K 45/06; A61K 31/4985; A61K 47/02; A61K 31/403; A61K 31/40; A61K 31/496; A61K 9/00; A61K 47/26

(52) U.S. Cl. CPC A61K 31/522; A61K 9/0053; A61K 9/08; A61K 45/06; A61K 47/26; A61K 47/02; A61K 31/403; A61K 31/40; A61K 31/496; A61K 31/4985

(57) ABSTRACT

The present invention relates to oral liquid pharmaceutical compositions comprising a DPP-IV inhibitor and processes for preparing the oral liquid pharmaceutical compositions. The invention also relates to oral liquid pharmaceutical compositions comprising a DPP-IV inhibitor and processes for preparing the oral liquid pharmaceutical compositions devoid of sugar based sweeteners. It further relates to a method of treating diabetes by administering said oral liquid pharmaceutical compositions.

### FIELD OF THE INVENTION

The present invention relates to oral liquid pharmaceutical compositions comprising a DPP-IV inhibitor and processes for their preparation. It further relates to a method of treating diabetes by administering said oral liquid pharmaceutical compositions.

### BACKGROUND OF THE INVENTION

DPP-IV (dipeptidyl peptidase IV) is an enzyme that catalyzes the conversion of glucagon like peptide-1 (GLP-1) from its active form to its inactive form. DPP-IV inhibitors, also commonly known as “gliptins”, competitively inhibit the enzyme DPP-IV, thereby increasing the endogenous concentration of GLP-1, which further augments insulin secretion and improves the glycemic profile of patients with diabetes.

Presently, DPP-IV inhibitors such as sitagliptin, vildagliptin, saxagliptin, teneligliptin, alogliptin, and linagliptin are available as conventional tablet dosage forms. Although conventional tablet dosage forms constitute a preferred route of administration, certain group of patients, including geriatric patients and patients having dysphagia, i.e., difficulty in swallowing, face problems while taking these dosage forms. Further, as antidiabetic drugs are being prescribed for chronically, such a problem would lead to a high level of patient non-compliance. In view of this, oral liquid compositions provide the best alternative over conventional tablet dosage forms. Apart from achieving better patient compliance, oral liquid compositions offer unique advantages such as more reproducible bioavailability, rapid absorption from the gastrointestinal tract, and an option of a flexible dosing regimen based on body weight or body surface area. As DPP-IV inhibitors are generally bitter in taste, it remains a challenge to the formulators to design a palatable oral liquid formulation of DPP-IV inhibitors.

Further, there exists a need in the art to provide oral liquid compositions of a DPP-IV inhibitor devoid of a sugar-based sweetener and yet keeping the composition palatable.

The present inventors have now for the first time, developed palatable oral liquid pharmaceutical compositions comprising a DPP-IV inhibitor. Further the present invention also provides for the first time palatable oral liquid pharmaceutical compositions comprising a DPP-IV inhibitor which are free of sugar-based sweeteners.

### SUMMARY OF THE INVENTION

The present invention provides palatable oral liquid pharmaceutical compositions comprising a DPP-IV inhibitor. It further provides palatable oral liquid pharmaceutical compositions comprising a DPP-IV inhibitor which are free of a sugar-based sweetener. It further provides processes for preparing said oral liquid pharmaceutical compositions. It also provides a method of treating diabetes by administering said oral liquid pharmaceutical compositions.

### DETAILED DESCRIPTION OF THE INVENTION

A first aspect of the present invention provides a palatable oral liquid pharmaceutical composition comprising a DPP-IV inhibitor and one or more pharmaceutically acceptable excipients.

According to one embodiment of this aspect, there is provided a palatable oral liquid pharmaceutical composition which is free of a sugar-based sweetener comprising a DPP-IV inhibitor and one or more pharmaceutically acceptable excipients.

According to another embodiment of this aspect, there is provided a palatable oral liquid pharmaceutical composition which is free of a sugar-based sweetener, comprising a DPP-IV inhibitor and one or more pharmaceutically acceptable excipients, wherein the pharmaceutically acceptable excipients are selected from the group comprising of sweeteners, buffering agents, antioxidants, preservatives, chelating agents, thickening agents, wetting agents, pH-adjusting agents, solvents, coloring agents, flavoring agents, and combinations thereof.

According to another embodiment of this aspect, there is provided a palatable oral liquid pharmaceutical composition which is free of a sugar-based sweetener comprising a DPP-IV inhibitor and one or more pharmaceutically acceptable excipients, wherein the sweeteners are selected from the group comprising of a sugar alcohol, a non-nutritive sugar-based sweetener, and combinations thereof.

According to another embodiment of this aspect, there is provided a palatable oral liquid pharmaceutical composition which is free of a sugar-based sweetener, comprising a DPP-IV inhibitor and one or more pharmaceutically acceptable excipients, wherein the pH of the composition is in the range of about 3 to about 8.

According to another embodiment of this aspect, there is provided a palatable oral liquid pharmaceutical composition which is free of a sugar-based sweetener, comprising a DPP-IV inhibitor and one or more pharmaceutically acceptable excipients, wherein the composition is stable.

According to another embodiment of this aspect, there is provided a palatable oral liquid pharmaceutical composition which is free of a sugar-based sweetener, comprising a DPP-IV inhibitor and one or more pharmaceutically acceptable excipients, wherein the composition further comprises one or more additional antidiabetic drugs.

A second aspect of the present invention provides a process of preparing a palatable oral liquid pharmaceutical composition, comprising a DPP-IV inhibitor, wherein the process comprises the steps of:
(i) preparing a solution of a buffering agent in a solvent;
(ii) dissolving a DPP-IV inhibitor and one or more pharmaceutically acceptable excipients into the solution of step (i); and
(iii) adjusting the pH of the solution of the step (ii) with a pH-adjusting agent.

According to another embodiment of this aspect, there is provided a process of preparing a palatable oral liquid pharmaceutical composition which is free of a sugar-based sweetener, comprising a DPP-IV inhibitor, wherein the process comprises the steps of:
(iv) preparing a solution of a buffering agent in a solvent;
(v) dissolving a DPP-IV inhibitor and one or more pharmaceutically acceptable excipients into the solution of step (i); and
(vi) adjusting the pH of the solution of the step (ii) with a pH-adjusting agent.

A third aspect of the present invention provides a method of treating diabetes by administering a palatable oral liquid pharmaceutical composition which is free of a sugar-based sweetener comprising a DPP-IV inhibitor and one or more pharmaceutically acceptable excipients.

According to one embodiment of this aspect, there is provided a method of treating diabetes by administering a palatable oral liquid pharmaceutical composition which is free of a sugar-based sweetener, comprising a DPP-IV inhibitor and one or more pharmaceutically acceptable excipients, wherein the method comprises sequential or simultaneous administration of one or more additional antidiabetic drugs.

The term “palatable”, as used herein, means that the taste of the composition is such that there is a voluntary acceptance or ingestion of a composition.

The term “sugar-based sweetener”, as used herein, refers to a sweetener which is rich in carbohydrates. Suitable examples of sugar-based sweeteners are sucrose, fructose, lactose, maltose, ribose, galactose, dried glucose syrup, grape sugar, arabinose, raffinose, mannose, rhamnose, iso-maltose, gentiobiose, trehalose, cellobiose, neohesperidose, maltotriose, panose, neokestose, stachyose, and the like.

Suitable sugar alcohols are selected from the group comprising of erythritol, threitol, ribitol, arabinitol, xylitol, allitol, dulcitol, glucitol, sorbitol, mannitol, altritol, iditol, maltitol, lactitol, isomalt, and combinations thereof.

Non-nutritive sugar based sweeteners are sugar substitutes having a sweet taste but are non-caloric. Suitable non-nutritive sugar based sweeteners are selected from the group comprising of aspartame, alitame, acesulfame-K, cyclamate, stevioside, glycyrrhizin, sucralose, neohesperidin, dihydrochalcone, thaumatin, sodium saccharin, and combinations thereof.

The palatable liquid pharmaceutical composition of the present invention comprises sugar alcohols and/or non-nutritive sugar based sweeteners in an amount ranging from about 20% w/v to about 80% w/v of the total composition.

The term “about”, as used herein, refers to any value which lies within the range defined by a variation of up to ±10% of the value.

The term “stable”, as used herein, refers to chemical stability, wherein not more than 5% w/w of total related substances are formed on storage at 40° C. and 75% relative humidity or at 25° C. and 60% relative humidity for a period of at least three months to the extent necessary for the sale and use of the composition.

The term “DPP-IV inhibitors”, as used herein, refers to the class of drugs that exhibit inhibition of the DPP-IV enzyme. Suitable examples of DPP-IV inhibitors are selected from the group comprising of sitagliptin, vildagliptin, saxagliptin, teneligliptin, alogliptin, linagliptin, and pharmaceutically acceptable salts or esters thereof. The pharmaceutically acceptable salts or esters may be prepared from an inorganic acid or an organic acid selected from the group comprising of hydrochloric acid, hydrobromic acid, hydroiodic acid, nitric acid, carbonic acid, bicarbonic acid, sulfuric acid, phosphoric acid, bisulphonic acid, oxalic acid, formic acid, acetic acid, propionic acid, succinic acid, glycolic acid, gluconic acid, lactic acid, malic acid, tartaric acid, citric acid, ascorbic acid, glucuronic acid, maleic acid, fumaric acid, pyruvic acid, aspartic acid, glutamic acid, benzoic acid, anthranilic acid, mesylic acid, salicyclic acid, p-hydroxybenzoic acid, phenylacetic acid, mandelic acid, embonic acid, methanesulfonic acid, ethanesulfonic acid, benzenesulfonic acid, pantothenic acid, 2-hydroxyethanesulfonic acid, toluenesulfonic acid, sulfanilic acid, cyclohexylaminosulfonic acid, stearic acid, alginic acid, salicyclic acid, galactaric acid, and galacturonic acid. DPP-IV inhibitors as used in the compositions of the present invention may be present as crystalline, amorphous, anhydrous, hydrous, solvates, prodrugs, chelates, or complex forms. The dose of any of the DPP-IV inhibitors may depend upon the individual drug used in the liquid pharmaceutical composition of the present invention.

The term “liquid pharmaceutical composition”, as used herein, refers to solutions, suspensions, reconstitutable suspensions, elixirs, or emulsions. Preferably, the liquid pharmaceutical composition of the present invention is a solution.

Suitable additional antidiabetic drugs are selected from the group comprising of acarbose, miglitol, repaglinide, nateglinide, glibenclamide, gliclazide, glimepiride, glipizide, tolbutamide, metformin, phenformin, rosiglitazone, pioglitazone, troglitazone, farglitazar, englitozone, darglitazone, isaglitazone, reglitazar, rivoglitazone, liraglutide, muraglitazar, peliglitazar, tesaglitazar, canagliflozin, dapagliflozin, remogliflozin, sergliflozin, and pharmaceutically acceptable salts or esters thereof. Further, the additional antidiabetic drug may be a DPP-IV inhibitor.

Suitable buffering agents are selected from the group comprising of sodium bicarbonate, potassium bicarbonate, magnesium hydroxide, magnesium lactate, aluminum hydroxide, sodium citrate, sodium tartarate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, sodium acetate, potassium metaphosphate, magnesium oxide, magnesium carbonate, magnesium silicate, calcium acetate, calcium glycerophosphate, calcium hydroxide, calcium lactate, calcium carbonate, calcium bicarbonate, and combinations thereof.

Suitable antioxidants are selected from the group comprising of butylated hydroxyl anisole, butylated hydroxyl toluene, tocopherol, ascorbyl palmitate, ascorbic acid, sodium metabisulfite, sodium sulfite, sodium thiosulfate, propyl gallate, and combinations thereof.

Suitable preservatives are selected from the group comprising of methyl paraben, ethyl paraben, propyl paraben, butyl paraben, benzoic acid, sodium benzoate, benzyl alcohol, sorbic acid, potassium sorbate, and combinations thereof.

Suitable chelating agents are selected from the group comprising of disodium EDTA, tartaric acid, malic acid, citric acid, and combinations thereof.

Suitable thickening agents are selected from the group comprising of hydroxy propylcellulose, sodium carboxy methylcellulose, xanthan gum, acacia, guar gum, locust bean gum, gum tragacanth, starch, carbopols, methylcellulose, polyvinylpyrrolidone, and combinations thereof.

Suitable wetting agents are selected from the group comprising of sodium lauryl sulphate, sorbitan esters of fatty acids, sorbitan monolaurate, sorbitan monooleate, sorbitan trioleate, sorbitan monopalmitate, sorbitan monostearate, sorbitan tristearate, ethylene oxide-propylene oxide block copolymers, lecithins, oleic acid and oleic acid salts, propylene glycol monostearate and monolaurate, glycerol monostearate and monooleate, fatty alcohol-polyethylene glycol ethers, fatty acid-polyethylene glycol esters, sodium dodecyl sulphate, dioctyl sodium sulphosuccinate, ethoxylated mono- and diglycerides, sucrose fatty acid esters, fatty acid salts, ethoxylated triglycerides, polyoxyethylated hydrogenated castor oil, sterol, and combinations thereof.

Suitable pH-adjusting agents are selected from the group comprising of hydrochloric acid, acetic acid, ammonia solutions, monoethanolamine, diethanolamine, triethanolamine, meglumine, sodium citrate, citric acid, lactic acid, phospharic acid, propionic acid, sulphiric acid, tartaric acid, potassium bicarbonate, potassium citrate, potassium hydroxide, sodium bicarbonate, sodium borate, and sodium hydroxide.

Suitable solvents are selected from the group comprising of water, propylene glycol, dipropylene glycol, polypropylene glycol, ethylene glycol, polyethylene glycol, glycerin, butylene glycol, hexylene glycol, polyoxyethylene, and combinations thereof.

Suitable coloring agents are selected from the group comprising of natural coloring agents; natural juice concentrates; pigments such as titanium dioxide, iron oxide, and zinc oxide; and combinations thereof.

Suitable flavoring agents are selected from the group comprising of grapefruit, orange, lime, lemon, mandarin, pineapple, strawberry, raspberry, mango, passion fruit, kiwi, apple, pear, peach, apricot, cherry, grapes, banana, cranberry, blueberry, black currant, red currant, gooseberry, lingonberries, cumin, thyme, basil, camille, valerian, fennel, parsley, camomile, tarragon, lavender, dill, bargamot, salvia, aloe vera balsam, spearmint, peppermint, eucalyptus, and combinations thereof.

The following examples represent various embodiments according to the present invention. The examples are given solely for the purpose of illustration and are not to be construed as limitations of the present invention, as many variations thereof are possible without departing from the spirit and scope of the invention.

### EXAMPLES

### Example 1

| Ingredients | Quantity (% w/v) |
|---|---|
| Linagliptin | 0.10 |
| Xylitol | 45.00 |
| Potassium bicarbonate | 0.50 |
| Hydrochloric acid | 0.63 |
| Sucralose | 0.20 |
| Strawberry flavor | 0.25 |
| Purified water | q.s. |

### Procedure:

1. Potassium bicarbonate was dissolved in purified water.

2. Linagliptin was added to the solution of step 1 and dissolved.

3. Hydrochloric acid was added into the solution of step 2.

4. Xylitol was dissolved into the solution of step 3.

5. Strawberry flavor was added into the solution of step 4.

6. Sucralose was dissolved in water and the solution of sucralose was added into the solution of step 5.

7. The pH of the solution of step 6 was adjusted to 4 to 6 using hydrochloric acid.

8. Purified water was added to achieve the desired volume.

### Example 2

| Ingredients | Quantity (% w/v) |
|---|---|
| Saxagliptin | 0.10 |
| Xylitol | 45.00 |
| Potassium bicarbonate | 0.50 |
| Hydrochloric acid | 0.63 |
| Sucralose | 0.20 |
| Strawberry flavor | 0.25 |
| Purified water | q.s. |

### Procedure:

1. Potassium bicarbonate was dissolved in purified water.

2. Saxagliptin was added to the solution of step 1 and dissolved.

3. Hydrochloric acid was added into the solution of step 2.

4. Xylitol was dissolved into the solution of step 3.

5. Strawberry flavor was added into the solution of step 4.

6. Sucralose was dissolved in water and the solution of sucralose was added into the solution of step 5.

7. The pH of the solution of step 6 was adjusted to 4 to 6 using hydrochloric acid.

8. Purified water was added to achieve the desired volume.

### Example 3

| Ingredients | Quantity (% w/v) |
|---|---|
| Vildagliptin | 0.50 |
| Xylitol | 45.00 |
| Potassium bicarbonate | 0.50 |
| Hydrochloric acid | 0.63 |
| Sucralose | 0.20 |
| Strawberry flavor | 0.25 |
| Purified water | q.s. |

### Procedure:

1. Potassium bicarbonate was dissolved in purified water.

2. Vildagliptin was added to the solution of step 1 and dissolved.

3. Hydrochloric acid was added into the solution of step 2.

4. Xylitol was dissolved into the solution of step 3.

5. Strawberry flavor was added into the solution of step 4.

6. Sucralose was dissolved in water and the solution of sucralose was added into the solution of step 5.

7. The pH of the solution of step 6 was adjusted to 4 to 6 using hydrochloric acid.

8. Purified water was added to achieve the desired volume.

### Example 4a and Example 4b

Quantity (% w/v)

| Ingredients | Example 4a | Example 4b |
|---|---|---|
| Teneligliptin hydrobromide | 0.20 | 0.40 |
| Xylitol | 45.00 | 45.00 |
| Potassium bicarbonate | 0.50 | 0.50 |
| Hydrochloric acid | 0.63 | 0.63 |
| Sucralose | 0.20 | 0.20 |
| Strawberry flavor | 0.25 | 0.25 |
| Purified water | q.s. | q.s. |

### Procedure:

1. Potassium bicarbonate was dissolved in purified water.

2. Teneligliptin hydrobromide was added to the solution of step 1 and dissolved.

3. Hydrochloric acid was added into the solution of step 2.

4. Xylitol was dissolved into the solution of step 3.

5. Strawberry flavor was added into the solution of step 4.

6. Sucralose was dissolved in water and the solution of sucralose was added into the solution of step 5.

7. The pH of the solution of step 6 was adjusted to 4 to 6 using hydrochloric acid.

8. Purified water was added to achieve the desired volume.

### Example 5a and Example 5b

Quantity (% w/v)

| Ingredients | Example 5a | Example 5b |
|---|---|---|
| Sitagliptin phosphate | 1.00 | 2.50 |
| Xylitol | 45.00 | 45.00 |
| Potassium bicarbonate | 0.50 | 0.50 |
| Hydrochloric acid | 0.63 | 0.63 |
| Sucralose | 0.20 | 0.20 |
| Strawberry flavor | 0.25 | 0.25 |
| Purified water | q.s. | q.s. |

### Procedure:

1. Potassium bicarbonate was dissolved in purified water.

2. Sitagliptin phosphate was added to the solution of step 1 and dissolved.

3. Hydrochloric acid was added into the solution of step 2.

4. Xylitol was dissolved into the solution of step 3.

5. Strawberry flavor was added into the solution of step 4.

6. Sucralose was dissolved in water and the solution of sucralose was added into the solution of step 5.

7. The pH of the solution of step 6 was adjusted to 4 to 6 using hydrochloric acid. 8. Purified water was added to achieve the desired volume.

We claim:

1. A palatable oral liquid pharmaceutical composition comprising a DPP-IV inhibitor and one or more pharmaceutically acceptable excipients.

2. A palatable oral liquid pharmaceutical composition devoid of a sugar-based sweetener, comprising a DPP-IV inhibitor and one or more pharmaceutically acceptable excipients.

3. The palatable oral liquid pharmaceutical composition according to claim 1 or 2, wherein the pharmaceutically acceptable excipients are selected from the group comprising of sweeteners, buffering agents, antioxidants, preservatives, chelating agents, thickening agents, wetting agents, pH-adjusting agents, solvents, coloring agents, flavoring agents, and combinations thereof.

4. The palatable oral liquid pharmaceutical composition according to claim 3, wherein the sweetener is selected from the group comprising of a sugar alcohol, a non-nutritive sugar based sweetener, and combinations thereof.

5. The palatable oral liquid pharmaceutical composition according to claim 4, wherein the sugar alcohols are selected from the group comprising of erythritol, threitol, ribitol, arabinitol, xylitol, allitol, dulcitol, glucitol, sorbitol, mannitol, altritol, iditol, maltitol, lactitol, isomalt, and combinations thereof.

6. The palatable oral liquid pharmaceutical composition according to claim 5, wherein the non-nutritive sugar based sweeteners are selected from the group comprising of aspartame, alitame, acesulfame-K, cyclamate, stevioside, glycyrrhizin, sucralose, neohesperidin, dihydrochalcone, thaumatin, sodium saccharin, and combinations thereof.

7. The palatable oral liquid pharmaceutical composition according to claim 1 or 2, wherein the composition is selected from the group comprising of solutions, suspensions, reconstitutable suspensions, elixirs, and emulsions.

8. The palatable oral liquid pharmaceutical composition according to claim 7, wherein the composition is a solution.

9. The palatable oral liquid pharmaceutical composition according to claim 1 or 2, wherein the pH of the composition is in the range of about 3 to about 8.

10. The palatable oral liquid pharmaceutical composition according to claim 1 or 2, wherein said composition is stable during storage at 40° C. and 75% relative humidity; and
25° C. and 60% relative humidity for a period of at least three months.

11. The palatable oral liquid pharmaceutical composition according to claim 1 or 2, wherein the composition further comprises one or more additional antidiabetic drugs.

12. A process of preparing a palatable oral liquid pharmaceutical composition, according to claim 1 or 2, wherein the process comprises the steps of:
(i) preparing a solution of a buffering agent in a solvent;
(ii) dissolving a DPP-IV inhibitor and one or more pharmaceutically acceptable excipients into the solution of step (i); and
(iii) adjusting the pH of the solution of the step (ii) with a pH-adjusting agent.

13. A method of treating diabetes by administering a palatable oral liquid pharmaceutical composition, comprising a DPP-IV inhibitor and one or more pharmaceutically acceptable excipients.

14. A method of treating diabetes by administering a palatable oral liquid pharmaceutical composition devoid of a sugar-based sweetener, comprising a DPP-IV inhibitor and one or more pharmaceutically acceptable excipients.

15. The method of treating diabetes comprising administering a palatable oral liquid composition according to claim 11, wherein the method comprises sequential or simultaneous administration of one or more additional antidiabetic drugs.
